(19) United States
(12) Patent Application Publication
(10) Pub. No.: US 2005/0089587 A1
(43) Pub. Date: Apr. 28, 2005

(54) Procedure for the preparation of microbiological associations intended to strengthen the medicinal herb's therapeutic qualities

(76) Inventor: Hector Vargas-Garza, Nuevo Leon (MX)

Correspondence Address: BREINER & BREINER, 115 NORTH HENRY STREET, P. O. BOX 19290, ALEXANDRIA, VA, 22314, US

(21) Appl. No.: 10/691,622

(22) Filed: Oct. 24, 2003

(51) Int. Cl. A61K 35/78; A61K 45/00

(52) U.S. Cl. 424/729; 424/93.4

(57) ABSTRACT

This invention refers a procedure for the preparation of microbiological association to strengthen the medicinal herb's therapeutic qualities, through a mixture of black tea pineapple peel or other fruit, brown sugar and water obtaining a MICROBIOLOGICAL ASSOCIATION that will be used for that purpose.

### BACKGROWND OF THE INVENTION

The Use of medical herbs to heal diseases has being growing worldwide in the last years. Their heal effects are well known for the international scientific community. There are enough technical references that support the therapeutic effects in the human being, nevertheless, in its common use, do not allow to have immediately therapeutic effects, or almost null, also the way it is used have being indiscriminate without any support or technical references.

To obtain a better therapeutic efficiency it is necessary to use microbiological associations to strengthen the medicinal herb's therapeutic qualities.

The pulmonary tuberculosis disease has being increasing worldwide in the last years due to the microorganism's resistance (responsible for generate the disease), to the anti tuberculosis medicine.

Since 2001 I have being researching in Mexico with patients with pulmonary tuberculosis medicine resistance, within government and private medical institutions, using this microbiological associations with therapeutic quality medicine herbs, obtaining surprising favorable results thanks to the use of my invention.

### DESCRIPTION OF THE INVENTION

This invention has the objective of preparing microbiological associations to strengthen the medicinal herb's therapeutic qualities.

### THE BEST METHOD TO REALIZE THE INVENTION

To prepare the microbiological association that will strengthen the medicinal herb's therapeutic qualities, will need to:

To Prepare Black Tea:

to boil water in containers, add 25 small black tea bags (those used in a cup) to 17.5 liters of boiled water and 1,150 grams of brown sugar and let cool down at room temperature.

Production of the Microbiological Association:

Use the peel of ripe washed up pineapples (or some other fruits) and put it in a container with the pulp face down, add enough amount of cooled black tea, prepared previously, to cover totally the pineapple peel. Cover with a flannel and let it rest at 30° C. for 15 days or when it forms a thick layer in the surface. This layer I will call the MICROBIOLOGICAL ASSOCIATION.

Propagation of the Microbiological Association:

In a container use 2 kilograms of the MICROBIOLOGICAL ASSOCIATION and add 17.5 liters of the black tea previously prepared, let the mixture rest for 7 days at 30° C., obtaining a liquid and a solid mass containing the propagated MICROBIOLOGICAL ASSOCIATION ready for strengthen the medicinal herb's therapeutic qualities using several proprietary methods.

The obtained liquid and solid will be used in fermentation of medicinal herbs with other therapeutic qualities.

The previous description is offered as an example to illustrate the preferred method to realize the procedure, but not intended to limit the reach of this invention to what I have described, but to claim any method following the principles described to produce the mentioned results.

1. A procedure for preparing microbiological associations to strengthen the medicinal herb's therapeutic qualities, characterized by using black tea prepared with 17.5 liters of boiling water and 25 small bags of black tea and 1,150 grams of brown sugar and letting it cool down at room temperature.

2. A procedure for preparing microbiological associations to strengthen the medicinal herb's therapeutic qualities, as described in claim 1, characterized also for the production of a MICROBIOLOGICAL ASSOCIATION using the peel of ripe washed up pineapples (or some other fruits) and putting them in a container with the pulp face down, add enough amount of cooled black tea prepared previously to cover totally the pineapple peel; covering with a flannel and letting it rest at 30° C. for 15 days or when it forms a thick layer in the surface called MICROBIOLOGICAL ASSOCIATION.

3. A procedure for preparing microbiological associations to strengthen the medicinal herb's therapeutic qualities, as described in claim 1, characterized also for the PROPAGATION OF THE MIROBIOLOGICAL ASSOCIATION by the mixture of 2 kilograms of the previously prepared MICROBIOLOGICAL ASSOCIATION and adding 17.5 liters of the black tea previously prepared, letting the mixture rest for 7 days at 30° C., obtaining a liquid and a solid mass containing the propagated MICROBIOLOGICAL ASSOCIATION ready for strengthen the medicinal herb's therapeutic qualities using several proprietary methods.

4. A procedure for preparing microbiological associations to strengthen the medicinal herb's therapeutic qualities, as described in claim 1, characterized also because the obtained liquid and solid will be used in fermentation of medicinal herbs with other therapeutic qualities.
